(19) United States
(12) Patent Application Publication
(10) Pub. No.: US 2021/0405989 A1
(43) Pub. Date: Dec. 30, 2021

(54) METHOD FOR TRANSFERRING A SOFTWARE INSTALLATION PROCEDURE ONTO A MEDICAL DEVICE

(71) Applicant: Siemens Healthcare GmbH, Erlangen (DE)

(72) Inventors: Friedrich HEGENDOERFER, Weilersbach (DE); Andre DE OLIVEIRA, Uttenreuth (DE); Daniel KEIM, Steinwiesen (DE); Roland EMRICH, Adelsdorf (DE); Marco MANCHINU, Spardorf (DE); Florian HAGER, Erlangen (DE); Martin KORINTH, Zorneding (DE)

(73) Assignee: Siemens Healthcare GmbH, Erlangen (DE)

(21) Appl. No.: 17/355,361

(22) Filed: Jun. 23, 2021

(30) Foreign Application Priority Data
Jun. 29, 2020 (DE) 10 2020 208 036.4

(51) Int. Cl. G06F 8/61; G16H 40/40; G06K 19/06

(52) U.S. Cl. CPC G06F 8/63; G16H 40/40; G06K 19/0614; G06K 19/06037; G06K 19/06028

(57) ABSTRACT

A method is for transferring a software installation procedure onto a medical device. In an embodiment, the method includes providing an image on a transmit system via a computing unit of the transmit system, the image including a visual depiction based on the installation procedure; optically transferring the image from the transmit system onto the medical device; and determining the installation procedure on the medical device based upon the visual depiction of the image via a computing unit of the medical device.

### PRIORITY STATEMENT

The present application hereby claims priority under 35 U.S.C. § 119 to German patent application number DE 102020208036.4 filed Jun. 29, 2020, the entire contents of which are hereby incorporated herein by reference.

### FIELD

Example embodiments of the invention generally relate to a method for transferring a software installation procedure onto a medical device.

### BACKGROUND

It is necessary in many fields of technology to run an installation procedure in order to install software on a device. Running the installation procedure makes it possible, for example, to install a new application on the device or to update an application which is already installed on the device or to improve data security of the device, etc. Such an installation procedure is frequently provided in a database to which the device has no direct access. In this case, the installation procedure must then in particular be transferred to the device. The device may in particular be a medical device. A medical device may in particular be a medical imaging system such as for example an ultrasound device, an X-ray device, a computed tomography (CT) device, a C-arm, a single-photon emission computed tomography (SPECT) device, a positron emission tomography (PET) device or a magnetic resonance tomography (MRI) device. Alternatively, a medical device may be a patient couch or an automated support system or a robotic system, etc. Inter alia for data protection reasons, a medical device frequently has no direct access to the database which provides the installation procedure.

It is known to carry out the transfer of the installation procedure in two steps. In a first step, in order to install software, a technician loads the installation procedure via an external device, which does have access to the database, from the database to a mobile storage device. Such a mobile storage device may be for example a USB stick or a CD or DVD. The external device may in particular be a personal computer (PC) or a laptop, etc. The technician then connects the mobile storage device to the medical device in order to transfer or load the installation procedure onto the medical device. Connect means in this connection that a physical connection is created between the mobile storage device and the medical device. Such a physical connection may be made for example via a USB port on the medical device or via a CD drive of the medical device. However, it is complicated to ensure that, on transfer of the installation procedure from the mobile storage device onto the medical device, no malware is transferred onto the medical device. The malware may here be any kind of software which is designed to damage or spy on etc. the medical device.

In addition, it often not possible for an on-site technician to install the software or carry out the installation procedure and transfer the installation procedure. In particular, the on-site technician often cannot ensure that no malware is transferred on transfer of the installation procedure via the mobile storage device. In particular, it is then often necessary for a service technician from the medical device manufacturer to make a visit. This is firstly costly and secondly restricts the availability of timeslots for using the medical device if a software installation appointment first has to be agreed.

Document EP 3 358 483 A1 describes a method by which a configuration change can be transferred optically from a monitored device to a monitoring device.

Data transfer via an animated quick response (QR) code is described in https://hub.packtpub.com/introducing-txqr-data-transfer-via-animated-qr-codes/.

### SUMMARY

At least one embodiment of the present invention provides a rapid, inexpensive and tamper-proof method for transferring a software installation procedure onto a medical device.

Embodiments of the present application are directed to a method for transferring a software installation procedure onto a medical device; a system for transferring a software installation procedure onto a medical device; a computer program product and a computer-readable storage medium. Advantageous further developments are presented in the claims and in the following description.

The manner of the implementation of the embodiments are described below with regard both to the apparatuses and to the method. Features, advantages or alternative embodiments mentioned in this connection are likewise also transferable to the other claimed subjects and vice versa. In other words, the substantive claims (e.g. directed to an apparatus) may also be further developed with the features which are described or claimed in connection with a method. The corresponding functional features of the method are here formed by corresponding substantive modules.

At least one embodiment of the invention relates to a method for transferring a software installation procedure onto a medical device. The method comprises a method step of providing an image on a transmit system via a computing unit of the transmit system. The image here comprises a visual depiction wherein the visual depiction is based on the installation procedure. The method further comprises a method step of optically transferring the image from the transmit system onto the medical device. The method further comprises a method step of determining the installation procedure on the medical device based upon the visual depiction of the image via a computing unit of the medical device.

At least one embodiment of the invention moreover relates to a system for transferring a software installation procedure onto a medical device. The system comprises a transmit system and a medical device. The transmit system comprises a computing unit for providing an image and a visual output unit for showing the image. The image here comprises a visual depiction. The visual depiction is here based on the installation procedure. The medical device here comprises a visual input unit for recording the image and a computing unit for determining the installation procedure based upon the visual depiction.

At least one embodiment of the invention also relates to a computer program product with a computer program and a computer-readable medium for a system. A largely software-based embodiment has the advantage that systems which are already in service can also straightforwardly be retrofitted to operate in the manner according to an embodiment of the invention via a software update. In addition to the computer program, such a computer program product may comprise additional elements such as for example documentation and/or additional components including hardware components, such as for example hardware keys (dongles etc.) for using the software.

At least one embodiment of the invention may also relate to a computer program product with a computer program which is directly loadable into a storage device of a system, with program parts for carrying out all the steps of the above-described method and the embodiments thereof when the program parts are run by the system.

At least one embodiment of the invention may also relate to a computer-readable storage medium on which program parts runnable by a system are stored in order to carry out all the steps of the above-described method and the embodiments thereof when the program parts are run by the system.

At least one embodiment of the invention may also relate to a method for transferring a software installation procedure onto a medical device, comprising:

providing an image on a transmit system via at least one processor of the transmit system, the image including a visual depiction, the visual depiction being based on the software installation procedure;

optically transferring the image from the transmit system onto the medical device; and

determining the software installation procedure on the medical device based upon the visual depiction of the image via at least one processor of the medical device.

At least one embodiment is further directed to a system for transferring a software installation procedure onto a medical device, comprising:
a transmit system; and
a medical device, wherein the transmit system comprises at least one processor to provide an image and a visual output unit to show the image, wherein the image includes a visual depiction,
wherein the visual depiction is based on the installation procedure, and
wherein the medical device includes a visual input unit to record the image and at least one processor to determine the installation procedure based upon the visual depiction.

At least one embodiment is further directed to such a system, further configured to carry out at least
providing an image on a transmit system via at least one processor of the transmit system, the image including a visual depiction, the visual depiction being based on the software installation procedure;
optically transferring the image from the transmit system onto the medical device; and
determining the software installation procedure on the medical device based upon the visual depiction of the image via at least one processor of the medical device.

At least one embodiment is further directed to a non-transitory computer program product storing a computer program, directly loadable into a storage device of a system, including program parts for carrying out at least one of the provisioning, optical transferring and determining of the method of an embodiment when the program parts are run by the system.

At least one embodiment is further directed to a non-transitory computer-readable storage medium storing program parts runnable by a system, to carry out at least one of the provisioning, optical transferring and determining of the method of an embodiment when the program parts are run by the system.

### BRIEF DESCRIPTION OF THE DRAWINGS

The above-described properties, features and advantages of this invention will be clearer and more readily comprehensible in connection with the following figures and the description thereof. The figures and description are not intended in any way to limit the invention and the embodiments thereof.

Identical components in different figures are provided with corresponding reference signs. The figures are not in general true to scale.

In the drawings

FIG. 1 shows a first example embodiment of the method according to the invention,

FIG. 2 shows a second example embodiment of the method according to the invention,

FIG. 3 shows a third example embodiment of the method according to the invention,

FIG. 4 shows a system comprising a transmit system and a medical device together with a database,

FIG. 5 shows a system comprising a transmit system and a medical device together with a database according to FIG. 4 additionally comprising an acoustic output unit and an acoustic input unit of the transmit system and an acoustic output unit and an acoustic input unit of the medical device,

FIG. 6 shows a two-dimensional barcode,

FIG. 7 shows a one-dimensional barcode, and

FIG. 8 shows an example embodiment of a system with holding apparatus.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for transferring a software installation procedure onto a medical device. The method comprises a method step of providing an image on a transmit system via a computing unit of the transmit system. The image here comprises a visual depiction wherein the visual depiction is based on the installation procedure. The method further comprises a method step of optically transferring the image from the transmit system onto the medical device. The method further comprises a method step of determining the installation procedure on the medical device based upon the visual depiction of the image via a computing unit of the medical device.

The medical device may in particular be a medical imaging system. The medical imaging system may in particular be an ultrasound device or an X-ray device or a computed tomography (CT) device or a C-arm or a single-photon emission computed tomography (SPECT) device or a positron emission tomography (PET) device or a magnetic resonance tomography (MRI) device etc. Alternatively, the medical device may for example be a patient couch or an automated support system or a robotic system. In particular, a medical device may be a device which optimizes and/or assists a workflow sequence in a medical environment.

The installation procedure in particular comprises an executable file. In particular, the executable file may be an .exe file. The installation procedure in particular comprises steps which are carried out for installing the software on the medical device. In particular, running the installation procedure installs the software.

The software may in particular be an application or computer program which can be installed on the medical device. In particular, the application may be run to control the medical device or to capture an image or to evaluate an image capture or to safety test the medical device etc. An application may in particular comprise a graphical user interface (GUI). The GUI may in particular be displayed to a user via a graphical output unit, for example a screen. The user may in particular be a member of medical personnel.

Alternatively or additionally, the software may be an update of an application. In this case, the application may already be installed on the medical device. The update may for example extend or improve an area of use of the application. In particular, the update may for example for this purpose add new functions to the application. Alternatively, the update may improve the application's security, in particular data security. Alternatively, the update may comprise a modification or update or adaptation of the GUI.

Alternatively or additionally, the software may comprise configuration data. The configuration data may be designed to configure the application. For an application for X-ray imaging, the configuration data may for example define an exposure time, an X-ray parameter, application of a filter etc. In particular, the configuration data may comprise standardized data for a standard examination. A standard examination may for example be a mammography or a thoracic X-ray or a head MRI etc.

The installation procedure may in particular be encoded in a visual depiction. This visual depiction may be comprised by an image. In other words, the image constitutes the visual depiction of the installation procedure. In particular, the image comprising the visual depiction may be transferred via electromagnetic radiation in the visible range. The electromagnetic radiation may in particular have a wavelength of between 200 nm and 800 nm. In particular, the electromagnetic radiation may have a wavelength of between 380 nm and 640 nm.

In the method step of providing the image, the image is provided on the transmit system. In particular, the installation procedure may be transferred for this purpose from an external database to the transmit system.

Alternatively, the transmit system can retrieve the installation procedure from an internal database. The installation procedure may here be transferred or retrieved in its “pure form”. In particular, the “pure form” of the installation procedure is written in a computer language or in a machine-readable code. The image comprising the visual information may then be determined on the transmit system. Alternatively, the installation procedure may be transferred or retrieved in the form of the image. Provision may in particular be initiated by user input. Alternatively, provision may proceed automatically as soon as a new installation procedure for the medical device is available in the database.

In the method step of optical transfer, the image is transferred from the transmit system onto the medical device. In particular, the image is transferred without physical contact between the transmit system and the medical device.

In the method step of determining the installation procedure, the installation procedure is decoded from the visual depiction. In other words, the installation procedure can be determined based upon the visual depiction via the computing unit.

The inventors have recognized that an installation procedure can be transferred without any physical connection between the transmit system and the medical device. They have recognized that the installation procedure can be encoded in a visual depiction for this purpose. This visual depiction can be optically transferred in the form of an image from the transmit system onto the medical device. The inventors have moreover recognized that it is in this manner possible to eliminate any need to physically connect an external device potentially contaminated with malware to the medical device in order to transfer the installation procedure. Any transfer of malware via the image can be virtually ruled out since the visual depiction would have to be deliberately modified for this purpose. The security (tamper resistance) of the medical system on transfer of an installation procedure can thus be improved. The inventors have moreover recognized that there is no need for a direct connection of the medical device to the database. This additionally improves the (data) security of the medical device. The inventors have moreover recognized that the method for transferring an installation procedure can be straightforwardly carried out by any user. There is thus no need for a service technician to make a visit to transfer the installation information. This saves costs and time.

According to one embodiment of the invention, provision of the image comprises retrieval of the installation procedure from a database.

In particular, the database may be an internal database of the transmit system. In particular, the internal database can be stored or saved on an internal storage device of the transmit system.

Alternatively, the database may be an external database. The external database may in particular be stored or saved on a server or in a cloud storage system. The transmit system may in particular access the external database via a network. The network may in particular be the internet or an intranet. The transmit system may be at least temporarily connected to the network via W-LAN (wireless local area network) and/or via LAN (local area network).

Alternatively, the transmit system may access the database via an external device. The database may for this purpose be stored or saved as an external database on an external device. Alternatively, the external device may access the external database via the network via LAN and/or W-LAN. In particular, the transmit system may be connected to the external device physically or via Bluetooth. In particular, the transmit system can retrieve the installation procedure via the external device or from the external device.

In some embodiments, the internal database can be synchronized with the external database via W-LAN and/or LAN and/or Bluetooth. The phrase “the transmit system has access to the database” hereinafter means that the transmit system can retrieve the installation procedure from the database.

In particular, on retrieval of the installation procedure, the installation procedure is (down)loaded from the database onto the transmit system. In other words, “retrieve” means “download”. Retrieval of the installation procedure may in particular comprise retrieval of the executable file or the pure or unmodified installation procedure. In particular, using the computing unit, the transmit system may then determine the visual depiction and the image of the installation procedure from the retrieved installation procedure. Alternatively, retrieval of the installation procedure may comprise retrieval of the visual depiction and the image. In particular, the visual depiction and thus the image is then determined in the database.

Retrieval of the installation procedure may in particular be initiated by the user. In particular, the user may obtain information that a new installation procedure is available in the database. The user may, for example, obtain the information by email and/or by SMS and/or by push message. Alternatively or additionally, the user may obtain the information via a servicing application from the manufacturer of the medical device. The servicing application may be installed on the transmit system.

Alternatively, retrieval of the installation procedure may proceed automatically when the transmit system has access to the database. In particular, an installation procedure which has not yet been retrieved from the transmit system can be automatically retrieved. In particular, retrieval may be associated with a user account. In particular, a user account may be created for the medical device. The user account may be saved in the database and/or on the transmit system. Information as to which installation procedure has already been retrieved and which installation procedure the user can prove to hold a license for can be saved in the user account. The user can purchase the license from the manufacturer of the medical device. An installation procedure which, according to the user account information has not yet been retrieved and for which a license is saved, can be automatically retrieved.

The inventors have recognized that it is not necessary for the medical device to have direct access to the database in order to transfer the installation procedure onto the medical device. The inventors have recognized that it is thus possible to avoid malware being directly transferred onto the medical device or information sensitive from a data protection standpoint being illegitimately accessed. The inventors have recognized that the installation procedure can be retrieved from the transmit system if the latter has at least temporary access to the database.

According to a further embodiment of the invention, the method step of optical transfer comprises the method steps of the image being shown by a visual output unit of the transmit system and of the image being recorded by a visual input unit of the medical device.

In particular, the visual output unit of the transmit system is configured to show or display the image. In particular, the visual output unit may take the form of a screen. The screen may in particular take the form of a field-emission display (FED) or a liquid crystal display (LCD) or a thin-film transistor display (TFT-LCD) or a cathode-ray tube display (CRT) or a plasma display or an organic light-emitting diode (OLED) or surface-conduction electron-emitter display (SED), etc.

The visual output unit may in particular be integrated into the transmit system. In other words, in particular the visual output unit and the computing unit of the transmit system may be integrated into one housing. In particular, the visual output unit and the computing unit are at least connected together in such a manner that data, in particular image data, can be transferred between the two units. Alternatively, the visual output unit may be separate from the computing unit. In other words, the visual output unit and the computing unit of the transmit system may be connected via an electrical connection, wherein the visual output unit and the computing unit are arranged spatially separately from one another. In particular, the connection may take the form of a cable for data transfer.

Alternatively, the visual output unit may take the form of a printer. In particular, the printer may be configured to print the image with the visual depiction on paper. The printer may in particular be a thermal printer or a dot-matrix printer or an inkjet printer or a laser printer etc.

Showing the image in particular involves showing the visual depiction comprised by the image.

The visual input unit of the medical device may in particular take the form of a camera. In particular, the camera may be configured to record or detect or capture optical information. In particular, the camera may be a digital camera. In particular, the camera may take the form of a video camera or a still camera. In particular, the camera can forward or transfer the recorded optical information in the form of pixel values. In particular, the camera can transfer the recorded optical information as graphics interchange format (GIF) or portable network graphics (PNG) or joint photographic experts group (JPG or JPEG) or tagged image file format (TIFF or TIF) or Windows bitmap (BMP) or audio video interleave (AVI) or moving picture experts group (MPEG), etc. In particular, the optical information is forwarded to the computing unit of the medical device. In particular, the visual input unit may be a camera which is standard equipment for the medical device. Alternatively, the camera may be additionally arranged on the medical device for this application. In particular, the visual input unit is connected to the computing unit of the medical device.

Alternatively, the visual input unit may be a scanner. In particular, the visual input unit may take the form of a scanner if the visual output unit takes the form of a printer. In particular, the scanner may be configured to scan the image printed on paper. In other words, the scanner may be configured to digitally record the image printed on paper.

In particular, the visual input unit may be integrated into the medical device. In other words, the visual input unit is then integrated into a housing of the medical device. In particular, the visual input unit and the computing unit of the medical device are at least connected together in such a manner that data, in particular image data, can be transferred between the two units. Alternatively, the visual input unit may be arranged separately from the medical device. In particular, the visual input unit is then only electrically connected to the computing unit of the medical device. In particular, the electrical connection may be configured for data transfer between the visual input unit and the computing unit of the medical device. In particular, the electrical connection may take the form of a cable.

In particular, during optical transfer, the visual output unit of the transmit system and the visual input unit of the medical device are oriented towards one another in such a manner that the visual input unit can record the image shown on the visual output unit. In particular, the image recorded in this manner can be transferred to the computing unit of the medical device.

The inventors have recognized that it is possible by optical transfer to avoid physical contact between the medical device and the transmit system or an alternative transfer system. The inventors have moreover recognized that any transfer of malware onto the medical device during optical transfer of the image can be virtually ruled out. The inventors have moreover recognized that the security of the data on the medical device can be ensured as well as possible in this way.

According to a further embodiment of the invention, the image comprises at least one first and one second image area. The at least one first image area here has a first color and the at least one second image area a second color. The first and second colors here differ at least in the lightness and/or the color shade thereof.

The lightness of a color may in particular denote the photometric lightness of the color. A color shade may in particular be stated in the “red-green-blue” model (RGB model). The first color and the second color may in particular in each case be a shade of gray. A shade of gray is obtained for example in the RGB model by all three color components (red, green and blue) having the same value. The first color may in particular be obtained in the RGB model by all the color components having a value of less than 50% of the maximum value, in particular of less than 10% of the maximum value, in particular of less than 1% of the maximum value and the first color may therefore in particular also be black. The second color may in particular be obtained in the RGB model by all the color components having a value of greater than 50% of the maximum value, in particular of greater than 90% of the maximum value, in particular of greater than 99% of the maximum value and the second color may therefore in particular also be white. The maximum value here denotes the maximum value of a color component.

Alternatively, the first and the second colors may in each case be any desired color shade. The color shade may be obtained by any desired combination of the three color components. In particular, the color shade of the first color and the color shade of the second color differ.

In particular, the image may have more than two image areas. Each image area here has its own color with its own color shade and/or lightness. In other words, the color of each individual image area differs from the colors of the other image areas.

In particular, an image area of one color may be non-contiguous in the image. In particular, an image area of one color may comprise more than one image sub-area which are non-contiguously distributed in the image. In other words, the image sub-areas of one color may be distributed in the image without any connection to one another.

The inventors have recognized that the installation information can be particularly efficiently encoded and transferred by such image areas.

According to a further embodiment of the invention, the visual depiction is present in a geometric arrangement of the at least one first and at least one second image areas.

In particular, each image area may comprise at least one image sub-area. In particular, each of the image areas may be subdivided into a plurality of image sub-areas or comprise a plurality of image sub-areas. The image sub-areas of an image area may here have the same lightness and/or the same color shade. The image sub-areas of different image areas may here have different lightness values and/or different color shades. In particular, an image sub-area may have a two-dimensional shape. In particular, an image sub-area may have a square or rectangular shape. In particular, the image sub-areas of all the image areas may be arranged in one dimension. In other words, the image sub-areas of all the image areas may be juxtaposed in one dimension. Alternatively, the image sub-areas may be arranged in two dimensions. In particular, the image sub-areas may be arranged in a square or rectangle. In other words, the image may take the form of a square or rectangle.

In particular, the arrangement of the image sub-areas of all the image areas describes the visual depiction of the installation procedure. In other words, the arrangement of the image areas describes the visual depiction of the installation procedure.

The inventors have recognized that such a geometric arrangement can be transferred particularly well, quickly and with particularly few errors from the transmit system onto the medical device.

According to a further embodiment of the invention, the visual depiction takes the form of at least one one-dimensional barcode or of at least one two-dimensional barcode.

A one-dimensional barcode may in particular be a linear barcode, while a two-dimensional barcode may in particular be a stacked linear barcode, a matrix code or a dot code. A two-dimensional barcode may in particular be a “quick response code” (QR code). A two-dimensional barcode may in particular alternatively be a “just another barcode” (JAB code). A JAB code is also denoted a multicolor QR code or polychrome QR code.

The visual depiction may in particular be a sequence of one-dimensional or two-dimensional barcodes. In particular, it may be a sequence of QR codes or JAB codes. Such a sequence of QR codes may be denoted an animated QR code.

A one-dimensional barcode or a two-dimensional barcode may in particular comprise redundant regions. In particular, the at least one first and the at least one second image area may in each case be such a region.

The inventors have recognized that information can be transferred particularly efficiently by a one-dimensional barcode or a two-dimensional barcode. Moreover, one-dimensional barcodes and two-dimensional barcodes comprise redundant regions such that errors during transfer can be prevented. At the same time, the redundant regions also accelerate optical transfer, since any inaccuracies in the positioning of the visual output unit relative to the visual input unit and in image recording by the visual input unit can be offset by the error redundancy. The inventors have moreover recognized that large volumes of data can be transferred via a JAB code and/or an animated QR code. In particular, the installation procedure frequently comprises such large volumes of data.

According to a further embodiment of the invention, the method moreover comprises the method steps of first sending of a first acoustic signal from an acoustic output unit of the transmit system and first reception of the first acoustic signal by an acoustic input unit of the medical device. The first acoustic signal here initiates optical transfer.

In particular, the first acoustic signal may be a signal of a specific audio frequency. In particular, the acoustic signal may be a signal in the audible frequency range. In particular, the audible frequency range may comprise frequencies between 16 Hz and 20 kHz. In particular, the first acoustic signal may be a whistle or beep of a specific audio frequency. Alternatively, the acoustic signal may be a sequence of tones. A sequence of tones may in particular be a sequence of more than one tone. In particular, the tones may have at least two different audio frequencies. Alternatively, the acoustic signal may be a signal in the ultrasound frequency range. In particular, the ultrasound frequency range comprises frequencies which are greater than 20 kHz.

In particular, the acoustic output unit of the transmit system may be a loudspeaker. In particular, the loudspeaker may be configured to output the first acoustic signal. In particular, the loudspeaker may be configured to output frequencies in the frequency range in which the first acoustic signal is located. In particular, the loudspeaker is configured to convert an electrical signal into a sound pressure or sound waves. The sound pressure here effects the first acoustic signal.

In particular, the acoustic output unit of the transmit system may be integrated into the transmit system. In particular, the acoustic output unit of the transmit system may be integrated into the housing of the transmit system. Alternatively, the acoustic output unit of the transmit system may be a separate component. In particular, the acoustic output unit of the transmit system may be electrically connected to the computing unit of the transmit system. In particular, the electrical connection may be configured for data transfer. In particular, the electrical connection may take the form of at least one cable.

In particular, the acoustic input unit of the medical device may be a microphone. In particular, the microphone is configured to convert a sound pressure into an electrical signal. In particular, the microphone is configured to convert a sound pressure, which effects the first acoustic signal, into an electrical signal. In particular, the microphone is sensitive to the frequency range of the audio frequency of the first acoustic signal.

In particular, the acoustic input unit may be integrated into the medical device. In particular, the acoustic input unit of the medical device may be integrated into the housing of the medical device. Alternatively, the acoustic input unit of the medical device may be a separate component. In particular, the separate component may be connected to the computing unit of the medical device via an electrical connection. In particular, the electrical connection may be configured for data transfer. In particular, the electrical connection may take the form of at least one cable.

In particular, the acoustic output unit of the transmit system sends the first acoustic signal during the method step of first sending when the optical transfer is initiated. In particular, the first acoustic signal may be sent on initiation by the user. In particular, the user may position the transmit system and/or the medical device in such a manner that the visual input unit of the medical device can record the visual output unit of the transmit system. In particular, the image may then be shown by the visual output unit. In particular, the first acoustic signal may then be sent in order to signal to the medical device that optical transfer can begin. In particular, the first reception of the first acoustic signal can activate the visual input unit. In particular, the first reception of the first acoustic signal may initiate recording of the image.

The inventors have recognized that the visual input unit of the medical device can be activated by the first acoustic signal. The inventors have recognized that the visual input unit can in this manner be switched off if no optical transfer is carried out or the visual input unit is otherwise required. In particular, the inventors have recognized that wear and tear on the visual input unit can be reduced as a consequence. In particular, the inventors have recognized that electricity can be saved in this manner if the visual input unit is only activated when required.

According to a further embodiment of the invention, the method moreover comprises the method steps of second sending of a second acoustic signal from an acoustic output unit of the medical device and second reception of the second acoustic signal by an acoustic input unit of the transmit system. The second acoustic signal here signals successful completion of the optical transfer.

In particular, the second acoustic signal may be configured according to one of the above-described embodiments of the first acoustic signal.

In particular, the acoustic output unit of the medical device may be configured in a manner similar to one of the above-described embodiments of the acoustic output unit of the transmit system. In particular, the acoustic output unit of the medical device may be integrated into the medical device or take the form of a separate component.

In particular, the acoustic input unit of the transmit system may be configured similarly to one of the above-described embodiments of the acoustic input unit of the medical device. In particular, the acoustic input unit may be integrated into the transmit system or take the form of a separate component.

In particular, the acoustic output unit of the medical device and the acoustic input unit of the transmit system are coordinated with one another in such a manner that they respectively send and receive an acoustic signal in the same frequency range. In other words, the acoustic output unit of the medical device and the acoustic input unit of the transmit system are sensitive in the same frequency range. In particular, the second acoustic signal is in this frequency range. In other words, the acoustic output unit of the medical device, the acoustic input unit of the transmit system and the second acoustic signal are coordinated with one another.

In particular, second sending proceeds when optical transfer has been successful. In particular, optical transfer is successful when the image has been completely recorded by the visual input unit. In particular, this may be denoted successful completion of the optical transfer.

The inventors have recognized that the method steps of second sending and second reception enable the user to monitor the method step of optical transfer. In particular, the user can recognize when optical transfer does not occur. The user can then check whether for example the visual input unit and the visual output unit are oriented towards one another in such a manner that the image shown on the visual output unit can be recorded by the visual input unit. Moreover, the second acoustic signal signals to the transmit system that the image need no longer be shown. The inventors have moreover recognized that the visual depiction may comprise a sequence of QR codes or an animated QR code. In particular, the second acoustic signal may then signal to the visual output unit that the next QR code of the QR code sequence can be displayed.

According to a further embodiment of the invention, the method moreover comprises a method step of running the installation procedure on the medical device.

In particular, when the installation procedure is run, the software can be installed on the medical device. Alternatively, running may comprise a software update. In particular, the installation procedure is run by the computing unit of the medical device.

The inventors have recognized that the installation procedure encoded in the visual depiction can be run to install the software on the medical device.

At least one embodiment of the invention moreover relates to a system for transferring a software installation procedure onto a medical device. The system comprises a transmit system and a medical device. The transmit system comprises a computing unit for providing an image and a visual output unit for showing the image. The image here comprises a visual depiction. The visual depiction is here based on the installation procedure. The medical device here comprises a visual input unit for recording the image and a computing unit for determining the installation procedure based upon the visual depiction.

According to a further embodiment of the invention, the transmit system comprises an acoustic output unit which is configured for first sending of a first acoustic signal, and the medical device an acoustic input unit which is configured for first reception of the first acoustic signal. The first acoustic signal here initiates optical transfer.

According to a further embodiment of the invention, the medical device comprises an acoustic output unit for second sending of a second acoustic signal, and the transmit system an acoustic input unit for second reception of the second acoustic signal. The second acoustic signal here signals successful completion of the optical transfer.

According to a further embodiment of the invention, the system is configured to carry out the above-described method and the embodiments thereof.

In particular, the computing unit of the transmit system, the visual output unit of the transmit system, the computing unit of the medical device and the visual input unit of the medical device are configured to carry out the steps of the method and the embodiments thereof.

In particular, the acoustic output unit of the transmit system and the acoustic input unit of the medical device may be configured to carry out the steps of the above-described method and the embodiments thereof.

In particular, the acoustic output unit of the medical device and the acoustic input unit of the transmit system may be configured to carry out the steps of the above-described method and the embodiments thereof.

According to a further embodiment of the invention, the transmit system and/or the medical device take the form of a mobile device.

In other words, the transmit system and/or the medical device may be of mobile configuration.

In particular, the transmit system may be a mobile telephone, in particular a smartphone. Alternatively, the transmit system may be a tablet. Alternatively, the transmit system may be a laptop. Alternatively, the transmit system may comprise a sheet of paper on which the image has been printed.

In particular, the medical device may be of displaceable configuration. In particular, the medical device may be of displaceable configuration in a room. In particular, the medical device may be of displaceable configuration in a building or building area. In particular, the medical device may be of displaceable configuration on a rail system. Alternatively, the medical device may be of freely displaceable configuration. In particular, the medical device may be of autonomously displaceable configuration. In particular, the medical device may then be denoted a mobile device.

In particular, the transmit system and/or the medical device may be of mobile configuration such that the visual output unit and the visual input unit can be positioned in such a manner that the image shown by the visual output unit can be recorded by the visual input unit. In particular, the transmit system can only access the database at one location. In particular, the location may differ from an examination location where the medical device is typically positioned.

The inventors have recognized that it is possible for the database to be accessed spatially independently of an optical transfer location if the transmit system and/or the medical device is of mobile configuration. The inventors have moreover recognized that the transmit system can be used for transferring an installation procedure for various medical devices if it is of mobile configuration.

According to a further embodiment of the invention, the system comprises a holding apparatus. The holding apparatus is here configured to position the transmit system in such a manner that the visual output unit is arranged in a detection region of the visual input unit.

In particular, the detection region may be the region in which the visual output unit can be positioned such that the visual input unit can record the image. In particular, the detection region may be arranged frontally in front of the visual input unit. In particular, the detection region may be arranged 50 cm to 2 m, in particular 75 cm, in particular 1 m, in particular 1.50 m, in front of the visual input unit. “In front of” means in this case that the visual output unit is arranged relative to the visual input unit in such a manner that the visual input unit can record the image displayed by the visual output unit.

In particular, the holding apparatus can be arranged on the medical device. In particular, the holding apparatus can be connected to the medical device. In particular, the connection may be a plug-in connection or a screw connection or a clip connection or a brazed connection or a riveted connection etc. Alternatively, the holding apparatus may be positioned in front of or spatially independently of the medical device. In particular, the holding apparatus may be of mobile configuration. In particular, the holding apparatus may be positioned by the user depending on a position of the medical device. In particular, the holding apparatus may be of rollable configuration. Alternatively, the holding apparatus may be fixedly positioned. In particular, the holding apparatus may then be anchored in a substrate. In particular, the medical device may be anchored on the same substrate. In particular, anchored may mean placed, bolted, cast or concreted etc. in place.

In particular, the transmit system is positionable in the holding apparatus. In particular, the transmit system can be placed or laid or clamped in the holding apparatus. In particular, the holding apparatus may be configured to fit the transmit system. In other words, the holding apparatus may be configured in such a manner that the transmit system is positionable without play in the holding apparatus. In other words, the transmit system adopts a fixed position in the holding apparatus. In some embodiments, the transmit system is for this purpose clampable in place in the holding apparatus via a clamp. In particular, the clamp may be of undoable configuration. In particular, it may be a screw clamp. Alternatively, it may be a clamp which is based on a fixable and undoable spring system. Alternatively, the holding apparatus may comprise a recess which is adapted to the transmit system. In particular, the transmit system may be inserted or laid in the recess for positioning.

The inventors have recognized that a holding apparatus facilitates optical transfer. In particular, it is thus not necessary for the user to position the transmit system manually in the detection region of the visual input unit. The inventors have recognized that errors during optical transfer due to shaking or wobbling on the part of the user can be avoided in this manner.

At least one embodiment of the invention also relates to a computer program product with a computer program and a computer-readable medium for a system. A largely software-based embodiment has the advantage that systems which are already in service can also straightforwardly be retrofitted to operate in the manner according to an embodiment of the invention via a software update. In addition to the computer program, such a computer program product may comprise additional elements such as for example documentation and/or additional components including hardware components, such as for example hardware keys (dongles etc.) for using the software.

At least one embodiment of the invention may also relate to a computer program product with a computer program which is directly loadable into a storage device of a system, with program parts for carrying out all the steps of the above-described method and the embodiments thereof when the program parts are run by the system.

At least one embodiment of the invention may also relate to a computer-readable storage medium on which program parts runnable by a system are stored in order to carry out all the steps of the above-described method and the embodiments thereof when the program parts are run by the system.

FIG. 1 shows a first example embodiment of the method according to the invention.

The first method step of the first example embodiment is provision PROV of an image on a transmit system 200. The image here comprises a visual depiction of an installation procedure. In particular, the installation procedure is encoded in the visual depiction. The installation procedure may be run to install software. In particular, the software can be installable on a medical device 220. In particular, the software may comprise an application or an update of an application already installed on the medical device 220 or a configuration file for the medical device 220. In the provision PROV step, the transmit system 200 can determine the image from the installation procedure. For this purpose, the transmit system can retrieve the installation procedure from a database 240 or from an external device. In particular, the database 240 may be an internal database of the transmit system. Alternatively, the database 240 may be an external database which the transmit system 200 can access via a network 230. Alternatively, the transmit system 200 can retrieve the image from the database 240 or from an external device.

The second method step of the first example embodiment is optical transfer TM of the image from the transmit system 200 to the medical device 220. In the optical transfer TM method step, the image is transferred via an optical medium 210 from the transmit system 200 onto a medical device 220. In particular, no physical coupling of the transmit system 200 and the medical device 220 is necessary for this purpose.

The third method step of the first example embodiment is determination DET of the installation procedure on the medical device 220 via a computing unit 222 of the medical device 220. In the determination DET method step of the installation procedure, the visual depiction comprised by the image is decoded. In this manner, the installation procedure is determined in its “pure form”. In particular, the “pure form” of the installation procedure is written in a computer language or in a machine-readable code.

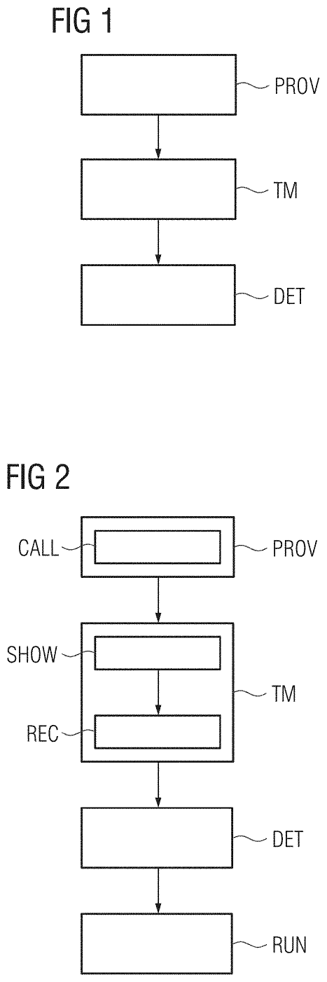

FIG. 2 shows a second example embodiment of the method according to the invention.

The second example embodiment is based on the first example embodiment according to FIG. 1.

The image provision PROV method step comprises a method step of retrieval CALL of the installation procedure from a database 240. The visual depiction comprised by the image is here based on the installation procedure. On retrieval CALL, the installation procedure is retrieved in its “pure form”. Alternatively, the image which comprises the visual depiction of the installation procedure can be retrieved. The database 240 may here in particular be an internal database 240 of the transmit system 200. In particular, the transmit system 200 can retrieve the installation procedure from the internal database 240. Alternatively, the database 240 may be an external database 240. In particular, the transmit system 200 may then access the database 240 via a network 230. In particular, network access may proceed via a LAN connection or a W-LAN connection. Access may in particular be location-independent. In other words, the transmit system 200 can access the database 240 from anywhere. Access may alternatively be location-dependent. In an alternative embodiment, the transmit system 200 may access the database 240 via an external device. The database 240 may here be saved on the external device. Alternatively, the external device may access the database 240 via the network 230. In particular, the transmit system 200 may be physically connected to the external device for this purpose. In particular, the transmit system 200 may be connected to the external device via USB or Bluetooth.

The optical transfer TM method step comprises the method steps of showing SHOW the image by a visual output unit 204 of the transmit system 200 and recording REC the image by a visual input unit 224 of the medical device 220.

The visual output unit 204 of the transmit system 200 may in particular be a screen or a monitor. In the showing SHOW method step, the image is shown or displayed or output on the visual output unit 204.

The visual input unit 224 of the medical device 220 may in particular be a camera. The camera may in particular take the form of a still camera or a video camera. In the image recording REC method step, the visual input unit 224 records the image shown by the visual output unit 204. For this purpose, the visual input unit 224 and the visual output unit 204 are positioned relative to one another in such a manner that the visual input unit 224 is arranged in a detection region of the visual output unit 204. The detection region is the region in which the visual input unit 224 can record the depiction of the image on the visual output unit 204.

The recording REC method step may proceed automatically or be initiated by a user or by the transmit system 200. In particular, the visual input unit 224 may automatically recognize when an image is displayed in its detection region. In particular, the visual input unit 224 may then automatically record the image. Alternatively, the user or the transmit system 200 may activate the visual input unit when the image is shown in the detection region. Initiation may in particular proceed via an optical signal or an acoustic signal or a mechanical signal. The mechanical signal may for example be actuation of a button or an input via a user interface of the medical device 220, etc.

The installation procedure determination DET method step is carried out in a manner similar to the description according to FIG. 1.

In a further method step, running RUN of the installation procedure proceeds on the medical device 220. Running RUN proceeds in particular via the computing unit 222 of the medical device 220. In particular, the installation procedure is here run in its “pure form”. In particular, running RUN of the installation procedure installs the software on the medical device 220.

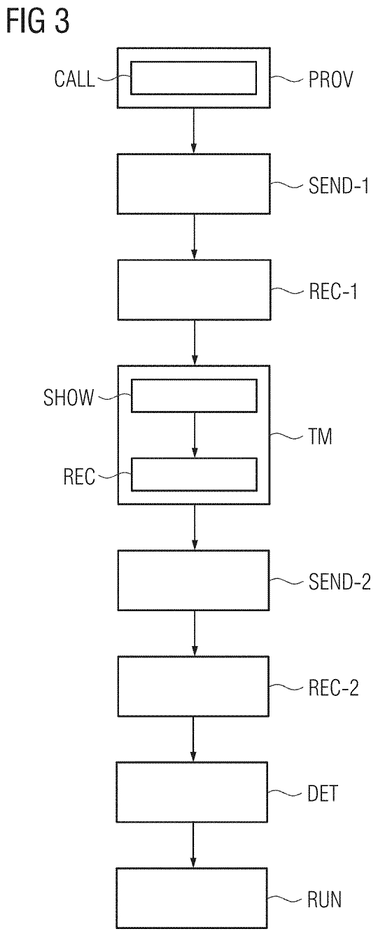

FIG. 3 shows a third example embodiment of the method according to the invention.

The method steps of providing PROV an image, retrieving CALL an installation procedure, optically transferring TM, showing SHOW the image, recording REC the image, determining DET the installation procedure and running RUN the installation procedure are carried out according to the description relating to FIGS. 2 and 3.

The method step of first sending SEND-1 of a first acoustic signal comprises a first acoustic signal being sent by an acoustic output unit 205 of the transmit system 200. The acoustic output unit 205 may in particular be a loudspeaker. The first acoustic signal may in particular be a tone of a fixed audio frequency. In particular, the audio frequency of the tone may be in the audible range or in the ultrasound range. Alternatively, the first acoustic signal may be a sequence of tones. The tones may here be of identical audio frequency. Alternatively, the tones may have different audio frequencies. In particular, the tones may be sent in a fixed rhythm. In particular, the acoustic output unit 205 of the transmit system is configured to convert an electrical signal into a sound wave or sound pressure. This sound pressure generates the tone.

The acoustic input unit 225 of the medical device 220 may in particular take the form of a microphone. In particular, the acoustic input unit 225 is configured to convert the sound pressure of a tone into an electrical signal. In particular, first reception REC-1 describes the conversion of the sound pressure of the tone or of the first acoustic signal, which is sent by the acoustic output unit 205 of the transmit system 200, into an electrical signal.

In particular, optical transfer TM can be initiated by the first sending SEND-1 and the first reception REC-1. In particular, the camera or the visual input unit 224 of the medical device 220 can for example be activated in this manner.

The method step of second sending SEND-2 of a second acoustic signal comprises the second acoustic signal being sent by an acoustic output unit 226 of the medical device. In particular, the acoustic output unit 226 of the medical device 220 may be configured in a manner similar to the acoustic output unit 205 of the transmit system 200.

In the method step of second reception REC-2, the second acoustic signal is received by an acoustic input unit 206 of the transmit system 200. In particular, the acoustic input unit 206 of the transmit system 200 may be configured in a manner similar to the acoustic input unit 225 of the medical device 220.

In particular, the second acoustic signal may be configured in a manner similar to the first acoustic signal. In particular, the second acoustic signal may comprise a different sequence of tones and/or a different audio frequency and/or a different rhythm from the first acoustic signal.

In particular, the second acoustic signal signals successful completion of the optical transfer TM. In particular, the second acoustic signal may deactivate the visual output unit 204. In particular, the second acoustic signal may indicate to a user that the optical transfer has been successful. In particular, the visual output unit 204 may then be removed from the detection region of the visual input unit 224. Alternatively, the second acoustic signal may signal that a subsequent QR code can be displayed on the visual output unit 204 if the visual depiction is an animated QR code.

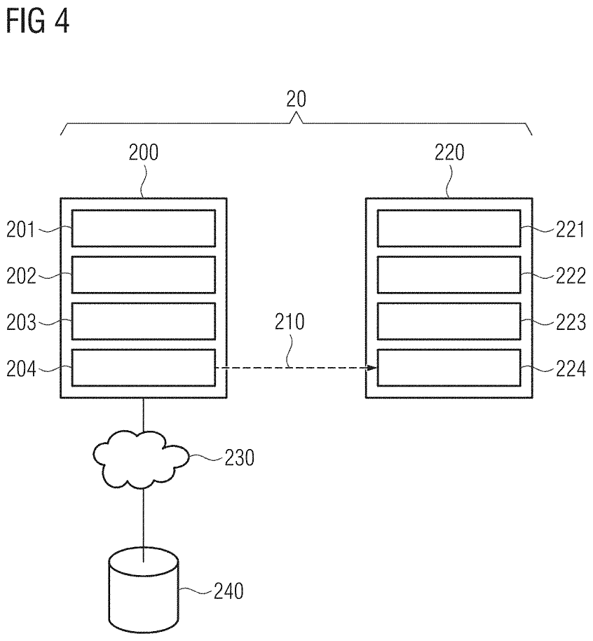

FIG. 4 shows a system 20 comprising a transmit system 200 and a medical device 220 together with a database 240. The transmit system here comprises an interface 201, a computing unit 202, a memory unit 203 and a visual output unit 204. The medical device 220 comprises an interface 221, a computing unit 222, a memory unit 223 and a visual input unit 224. The visual output unit 204 of the transmit system 200 is connected to the visual input unit 224 of the medical device 220 via an optical channel 210. The transmit system 200 is furthermore connected via a network 230 to the database 240. The transmit system 200 may in particular comprise a computer, a microcontroller or an integrated circuit, which comprise the interface 201, the computing unit 202, the memory unit 203 and/or the visual output unit 204. The medical device 220 may likewise comprise a computer, a microcontroller or an integrated circuit. The transmit system 200 may furthermore in particular be a mobile telephone, in particular a smartphone. Alternatively, the transmit system 200 may be a tablet. The interfaces 201, 221 may be a hardware or software interface (e.g. PCI bus, USB or FireWire). The computing unit 202 of the transmit system 200 and the computing unit 222 of the medical device 220 may have hardware or software elements, for example a microprocessor or a “field programmable gate array” (“FPGA”). The memory unit 203 of the transmit system 200 and the memory unit 223 of the medical device 220 may be embodied as a non-persistent working memory means (random access memory or RAM for short) or as a persistent mass storage device (hard disk, USB stick, SD card, solid-state disc). The visual output unit 204 may in particular be a screen but may alternatively also be a printer. The visual input unit 224 is in particular a camera. It may, however, also be a keyboard, a mouse or a scanner. The network 230 may be an intranet or in particular the internet. The database 240 may furthermore also comprise a computing unit for data management.

The visual input unit 224 of the medical device 220 is configured to record the visual depiction displayed by the visual output unit 204 of the transmit system 200. The visual depiction is here transferred via an optical medium 210, wherein the optical medium is in this case the ambient air. The visual depiction may here in particular be shown or encoded via an image, in particular via a QR code 600 or a linear barcode 700. The visual output unit 204 is for example, as in the example embodiment shown, a screen. In particular, the visual input unit 224 may take the form of a camera which can capture the QR code 600 shown on the screen. If the visual output unit 204 is a printer which prints paper with the visual depiction 700, the input unit 224 may likewise be a camera or a scanner.

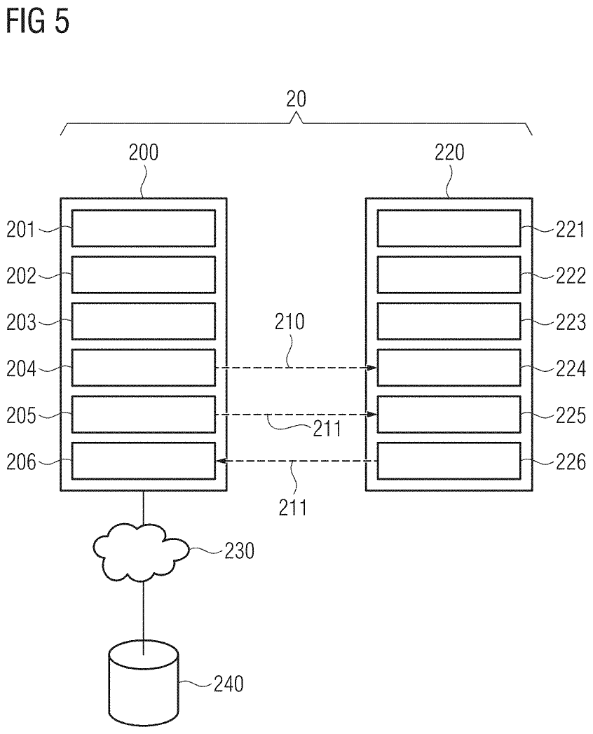

FIG. 5 shows a system 20 comprising a transmit system 200 and a medical device 220 together with a database 240 according to FIG. 4 additionally comprising an acoustic output unit 205 and an acoustic input unit 206 of the transmit system 200 and an acoustic output unit 226 and an acoustic input unit 225 of the medical device 220.

The acoustic output unit 205 of the transmit system 200 is in particular a loudspeaker. The loudspeaker is in particular configured to convert electrical information into sound waves or sound pressure. This sound pressure may be perceived as the first acoustic signal. In particular, the sound pressure can be transported by an acoustic medium 211. In particular, the acoustic medium 211 may be air. The acoustic input unit 225 of the medical device 220 may in particular be a microphone. The microphone may in particular be configured to receive the sound pressure sent by the acoustic output unit 205 of the transmit system 200 and convert it into an electrical signal.

In particular, the acoustic output unit 205 of the transmit system 200 may send the first acoustic signal when the optical transfer TM is initiated. In particular, the visual input unit 224 may be activated by first reception REC-1 of the acoustic signal by the acoustic input unit 225 of the medical device 220.

In particular, the acoustic input unit 206 of the transmit system 200 may be configured in a manner similar to the acoustic input unit 225 of the medical device 220. In particular, the acoustic output unit 226 of the medical device 220 may be configured in a manner similar to the acoustic output unit 205 of the transmit system 200.

In particular, the acoustic output unit 226 may send a second acoustic signal after successful completion of the optical transfer TM. In particular, the second reception REC-2 may signal to a user that the optical transfer TM has been successful.

In some embodiments, the system may only comprise the acoustic output unit 205 of the transmit system 200 and the acoustic input unit 225 of the medical device 220. In alternative embodiments, the system may only comprise the acoustic output unit 226 of the medical device 220 and the acoustic input unit 206 of the transmit system 200.

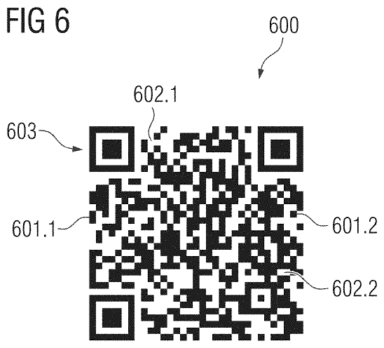

FIG. 6 shows a two-dimensional barcode 600. The two-dimensional barcode 600 is in this example embodiment a QR code 600. The QR code 600 consists of first image areas 601.1, 601.2, which are black in color, and second image areas 602.1, 602.2, which are white in color. In particular, the first image area comprises first image sub-areas 601.1, 601.2 and the second image area second image sub-areas 602.1, 602.2. The first image sub-areas 601.1, 601.2 are a cluster of black pixels, the second image sub-areas 602.1, 602.2 are a cluster of white pixels. The pixels of the QR code 600 are arranged in a grid structure. The QR code 600 furthermore comprises position markers 603 in the form of a geometric arrangement of white and black pixels; a QR code 600 may contain still further marks or patterns which facilitate recording the QR code 600 and/or decoding the QR code 600. The information or visual depiction of the QR code 600 is contained in the geometric shape of the first image areas or image sub-areas 601.1, 601.2 and the second image areas or image sub-areas 602.1, 602.2. The geometric shape of the first image areas or image sub-areas 601.1, 601.2 and the second image areas or image sub-areas 602.1, 602.2 is here provided in this example embodiment by contiguous square pixels in a grid.

A QR code 600 may contain redundant information. This redundant information may in particular be used to offset errors in the depiction or recording of the QR code 600. Various levels of redundancy are possible here, in particular more than 7% redundant content, in particular more than 15% redundant content, in particular more than 25% redundant content and in particular more than 30% redundant content. An item of content of the QR code 600 is here deemed redundant if the visual information contained in the QR code 600 can be determined even in the absence of this content. Reed-Solomon encoding is in particular known for implementing error correction.

In alternative embodiments, the QR code 600 may in particular take the form of a multicolor QR code or polychrome QR code or JAB code.

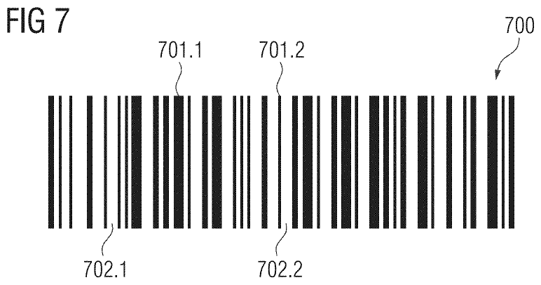

FIG. 7 shows a one-dimensional barcode 700. The one-dimensional barcode 700 is in this example embodiment a linear barcode 700. The linear barcode 700 consists of black lines 701.1, 701.2 and white lines 702.1 and 702.2. The black lines 701.1, 701.2 correspond to a first image area with image sub-areas. Each black line 701.1, 701.2 is here an image sub-area of the first image area. The white lines 702.1, 702.2 correspond to a second image area with image sub-areas. Each white line 702.1, 702.2 is here an image sub-area of the second image area. The information encoded in the linear barcode 700 is contained in the different thicknesses of the lines 701.1, 701.2, 702.1, 702.2. The linear barcode 700 may likewise contain redundant information, such that error correction on recording the linear barcode 700 is possible. In particular, it is known to use check characters.

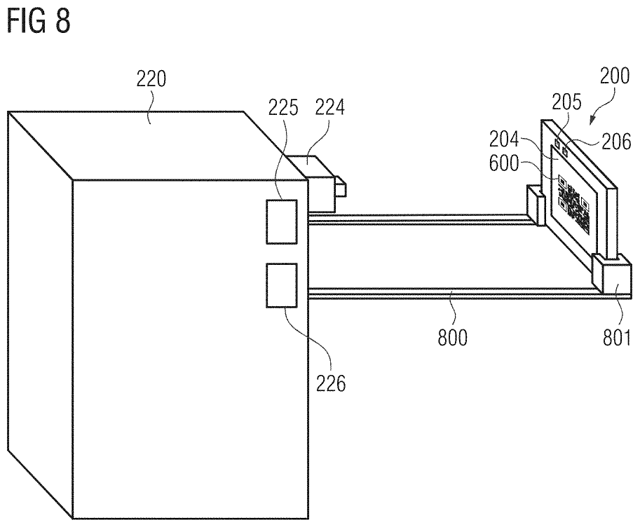

FIG. 8 shows an example embodiment of system 20 with holding apparatus 800.

The holding apparatus 800 is connected to the medical device 220. The holding apparatus 800 is in particular connected to the medical device 220 with a plug-in connection or a screw connection or a clip connection or a riveted connection or a brazed connection. The holding apparatus 800 comprises a clamp 801 or a clamp connection, with which the transmit system 200 can be fastened to or in the holding apparatus 800. In particular, the transmit system 200 may be fastened in such a manner that it is virtually motionless relative to the visual input unit 224 of the medical device 220. In particular, the transmit system 200 is positioned in the holding apparatus 800 in such a manner that the visual output unit 204 is arranged in a detection region of the visual input unit 224 in such a manner that the visual input unit 224 can record the image shown by the visual output unit 203. In this example embodiment, the image is a QR code 600. The acoustic output units 205, 226 and acoustic input units 206, 225 of the transmit system 200 and of the medical device 220 may be arranged at will on the respective device or system.

Where it has not yet been explicitly done but is reasonable and in line with the purposes of the invention, individual example embodiments, individual sub-embodiments or features thereof may be combined with one another or interchanged without going beyond the scope of the present invention. Advantages of the invention described in relation to one example embodiment also apply, where transferable, to other example embodiments without being explicitly stated to do so.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for transferring a software installation procedure onto a medical device, comprising:
providing an image on a transmit system via at least one processor of the transmit system, the image including a visual depiction, the visual depiction being based on the software installation procedure;
optically transferring the image from the transmit system onto the medical device; and
determining the software installation procedure on the medical device based upon the visual depiction of the image via at least one processor of the medical device.

2. The method of claim 1, wherein the providing of the image comprises retrieval of the installation procedure from a database.

3. The method of claim 1, wherein the optically transferring includes:
showing the image by a visual output unit of the transmit system, and
recording the image by a visual input unit of the medical device.

4. The method of claim 1,
wherein the image comprises at least one first image area and at least one second image area,
wherein the at least one first image area includes a first color and the at least one second image area includes a second color and
wherein the first color and the second color differ at least in at least one of the lightness and color shade.

5. The method of claim 4, wherein the visual depiction is present in a geometric arrangement of the at least one first image area and the at least one second image area of the image.

6. The method of claim 5, wherein the visual depiction takes the form of at least one one-dimensional barcode or at least one two-dimensional barcode.

7. The method of claim 1, wherein the method further comprises:
first sending of a first acoustic signal from an acoustic output unit of the transmit system, and
first reception of the first acoustic signal by an acoustic input unit of the medical device, the first acoustic signal initiating optical transfer.

8. The method of claim 1, wherein the method further comprises:
second sending of a second acoustic signal by an acoustic output unit of the medical device, and
second reception of the second acoustic signal by an acoustic input unit of the transmit system, wherein the second acoustic signal signals successful completion of the optical transfer.

9. The method claim 1, wherein the method further comprises: running the installation procedure on the medical device.

10. A system for transferring a software installation procedure onto a medical device, comprising:
a transmit system; and
a medical device, wherein the transmit system comprises at least one processor to provide an image and a visual output unit to show the image,
wherein the image includes a visual depiction,
wherein the visual depiction is based on the installation procedure, and
wherein the medical device includes a visual input unit to record the image and at least one processor to determine the installation procedure based upon the visual depiction.

11. The system of claim 10, further configured to carry out at least
providing an image on a transmit system via at least one processor of the transmit system, the image including a visual depiction, the visual depiction being based on the software installation procedure;
optically transferring the image from the transmit system onto the medical device; and
determining the software installation procedure on the medical device based upon the visual depiction of the image via at least one processor of the medical device.

12. The system of claim 10, wherein at least one of the transmit system and the medical device is in a form of a mobile device.

13. The system of claim 10, further comprising:
a holding apparatus configured to position the transmit system such that the visual output unit is arranged in a detection region of the visual input unit.

14. A non-transitory computer program product storing a computer program, directly loadable into a storage device of a system, including program parts for carrying out at least one of the provisioning, optical transferring and determining of the method of claim 1 when the program parts are run by the system.

15. A non-transitory computer-readable storage medium storing program parts runnable by a system, to carry out at least one of the provisioning, optical transferring and determining of the method of claim 1 when the program parts are run by the system.

16. The method of claim 2, wherein the optically transferring includes:
showing the image by a visual output unit of the transmit system, and
recording the image by a visual input unit of the medical device.

17. The method of claim 2,
wherein the image comprises at least one first image area and at least one second image area,
wherein the at least one first image area includes a first color and the at least one second image area includes a second color and
wherein the first color and the second color differ at least in at least one of the lightness and color shade.

18. The method of claim 17, wherein the visual depiction is present in a geometric arrangement of the at least one first image area and the at least one second image area of the image.

19. The method of claim 18, wherein the visual depiction takes the form of at least one one-dimensional barcode or at least one two-dimensional barcode.

20. The system of claim 10, wherein at least one of the transmit system and the medical device is in a form of a mobile device.
